(19) United States
(12) Patent Application Publication
(10) Pub. No.: US 2003/0092653 A1
(43) Pub. Date: May 15, 2003

(54) Method for inhibition of pathogenic microorganisms

(75) Inventors: Kevin Kisich, Lafayette, CO (US); Gill Diamond, Short Hills, NJ (US)

Correspondence Address: SHERIDAN ROSS PC, 1560 BROADWAY, SUITE 1200, DENVER, CO, 80202

(73) Assignee: National Jewish Medical & Research Center & University of Medicine & Dentistry of New Jersey.

(21) Appl. No.: 10/134,039

(22) Filed: Apr. 25, 2002

Related U.S. Application Data
(63) Continuation of application No. 09/672,723, filed on Sep. 28, 2000, now abandoned.
(60) Provisional application No. 60/157,348, filed on Sep. 30, 1999.

(51) Int. Cl. A61K 48/00; C12N 5/08

(52) U.S. Cl. 514/44; 435/366

(57) ABSTRACT

Disclosed is a method for inhibiting the growth of a microorganism by high efficiency transfection of a human host cell with a nucleic acid encoding an antimicrobial agent, such that the host cell expresses the antimicrobial agent effective to inhibit growth of the microorganism.

### CROSS-REFERENCE TO RELATED APPLICATIONS

This application claims priority under 35 U.S.C. § 119(e) from U.S. Provisional Application Serial No. 60/157,348, filed on Sep. 30, 1999, and entitled “A novel anti-mycobacterial agent based on mRNA encoding human β-defensin 2 enables primary macrophages to restrict growth of Mycobacterium tuberculosis.” The entire disclosure of U.S. Provisional Application Serial No. 60/157,348 is incorporated herein by reference.

### GOVERNMENT RIGHTS

This invention was made in part with government support under NIH Grant HL53400, awarded by the National Institutes of Health. The government has certain rights to this invention.

### FIELD OF THE INVENTION

This invention generally relates to a method for producing a therapeutic protein in a human host cell, and particularly, in a human primary macrophage. The invention also relates to a method to inhibit the growth of a pathogenic microorganism by expressing such a therapeutic protein in a human host cell.

### BACKGROUND OF THE INVENTION

Particular microorganisms have long been recognized as a source of disease. Pathogenic microorganisms cause disease by disrupting the normal functions of a host. Many pathogenic microorganisms, including intracellular bacteria, parasites, pathogenic yeast, and enveloped viruses, grow primarily in host cells where they are shielded from the effects of both antibodies and cytotoxic T cells. By developing ways to avoid the immune system, such microorganisms are able to multiply, and subsequently cause or contribute to inflammation and tissue damage in the infected organism.

As an example, tuberculosis (TB), caused by exposure to and infection with the mycobacterium, Mycobacterium tuberculosis, continues to infect and kill approximately 2 million people each year world wide. It is estimated that one out of three humans are infected, leading to 8,000,000 new cases of active tuberculosis each year (Dye et al., Jama, 282:677-86, 1999). TB is expected to double by the year 2020. Greater knowledge of the mechanisms of human resistance to this pathogen as well as new therapeutics are needed. One of the first cell types to encounter M. tuberculosis after inhalation of the organism is the macrophage. However, M. tuberculosis multiplies rapidly in cultured human macrophages even when they are stimulated with cytokines (Douvas et al., Infect Immun 50:1-8, 1985). Therefore, other elements of the immune system may assist macrophages in limiting the multiplication of tubercle bacilli in approximately one third of the earth's human population which is infected with M. tuberculosis, but does not develop active disease (Dye et al., Jama, 282:677-86, 1999).

Antimicrobial peptides are a recently discovered component of the innate immune system. They have been described in plants, tunicates, insects, fish, amphibia, and mammals, including humans, and are proposed to participate in the early host defense response against microorganisms. They are likely to be particularly important in the early phases of defense against invading microbes because they are available within minutes to hours after the first contact with the pathogen. Moreover, the peptides exhibit a broad spectrum of activity that includes bacteria, fungi and certain enveloped viruses. Antimicrobial peptides, which numbered greater than 100 as recently as 1998, can be classified based on structural features (See review in Hancock et al., 1995, Adv. Microb. Physiol. 37:135-175; Boman 1995, Annu. Rev. Immunol. 13:61-92; and Lehrer and Ganz, 1996, Ann. N.Y. Acad. Sci. 797:228-239). However, many of these different structural classes of peptides share certain common properties. These include cationic charge, a broad spectrum of antimicrobial activity via selective discretion of target membranes, and encoding by genes which are expressed with tissue specificity.

One important element of the human innate immune defenses against microorganisms are small antimicrobial peptides known as defensins (Ganz and Lehrer, Curr Opin Immunol 10:41-4, 1998). These small (30-50 aa) cationic peptides are found in a variety of mammalian myeloid and epithelial cells, and are bactericidal or bacteristatic for a broad spectrum of microbes, including Mycobacterium tuberculosis (Ogata et al., Infect. Immun. 60:4720-4725, 1992; Miyakawa et al., Infect. Immun. 64:926-932, 1996). Defensins are primarily divided into two subclasses, α- and β-defensins, based on structural characteristics, and are found in a variety of tissues and cell types. They are among the most abundant components in phagocytic cells, where they participate in the oxygen-independent killing of ingested microorganisms. In epithelial cells, such as the small intestinal crypts (Ouellette and Selsted, FASEB J. 10:1280-1289, 1996), female reproductive tract (Quayle et al., Am. J. Pathol. 152:1247-1258, 1998) and trachea (Diamond et al., Proc. Natl. Acad. Sci. (U.S.A.) 88:3952-3956, 1991), they have been predicted to provide a first line of host defense by acting in the luminal contents as a component of the innate immune response. In the mammalian airway, β-defensins have been found in tracheal mucosa (Diamond et al., Proc. Natl. Acad. Sci. (U.S.A.) 88:3952-3956, 1991), nasal secretions (Cole et al., Infect Immun 67:3267-75,1999) and brochoalveolar lavage fluid (Travis et al., Am J Respir Cell Mol Biol 20:872-9, 1999) at concentrations which are antimicrobial in vitro, suggesting that they can perform this function in vivo.

While defensins are found in rabbit (Patterson-Delafield et al., Infect Immun 31 :723-31, 1981) and bovine macrophages (Ryan et al., Infect. Immun. 66:878-881, 1998), they are absent from human macrophages (present inventors' unpublished data). Although defensins have been proposed for use as therapeutics (Ganz and Lehrer, Pharmacology & Therapeutics 66:191-205, 1995), chemical synthesis of these peptides is a challenge due to the complex pattern of disulfide bonds which stabilize the structure (Lauth et al., Insect Biochem Mol Biol 28:1059-66, 1998), and recombinant methods do not produce sufficient yields (Harwig et al., Meth. in Enzymol. 236:160-170, 1994; Valore and Ganz, Methods Mol Biol 78:115-31, 1997). An alternative to using defensin proteins as antimicrobial agents was described using DNA to encode the defensins for intracellular expression in a murine macrophage cell line, which resulted in greater resistance to Histoplasma capsulatum (Couto et al., Infection & Immunity 62:2375-8, 1994). To date, however, there are very few reports of primary human macrophage transfection with DNA plasmids. Moreover, those which quantitate transfection efficiency report that only about 2% of the cells express the reporter gene (eGFP) (Simoes et al., J Leukoc Biol 65:270-9, 1999; Van Tendeloo et al., Gene Ther 5:700-7, 1998; Weir and Meltzer, Cell Immunol 148:157-65, 1993).

Therefore, there remains a need in the art for a feasible method of producing and using therapeutic proteins such as defensins in human host cells which do not naturally express such proteins.

### SUMMARY OF THE INVENTION

One embodiment of the present invention relates to a method to inhibit the growth of a microorganism. Such a method includes the step of transfecting a human cell with an isolated mRNA encoding a protein having antimicrobial biological activity, wherein the human cell expresses the protein and thereby inhibits the growth of a microorganism when the microorganism contacts the human cell. The human cell is a natural host cell for the microorganism or naturally contacts the microorganism when a human is infected with the microorganism. In one aspect, the human cell does not naturally express the protein. In a preferred embodiment, the human cell is a primary macrophage. In one aspect, the primary human macrophage resides in lung tissue.

The microorganism which can be inhibited by the method of the present invention can be any microorganism that is susceptible to inhibition by an antimicrobial and particularly includes pathogenic microorganisms. Pathogenic microorganisms include, but are not limited to, a bacterium, a fungus, a virus, a protozoa and a parasite. Bacterium that may be inhibited using the present method include, but are not limited to: a spirochete, a mycobacterium, a Gram (+) cocci, a Gram (−) cocci, a Gram (+) bacillus, a Gram (−) bacillus, an anaerobic bacterium, a rickettsias, a Chlamydias and a mycoplasma. A preferred bacterium to inhibit using the present method is a mycobacterium. A fungus that may be inhibited using the present method include, but are not limited to: a pathogenic yeast, a mold and a dimorphic fungus. Preferred viruses to inhibit by the present method include enveloped viruses.

An antimicrobial protein produced by the present method can include any antimicrobial protein. In one embodiment, the antimicrobial protein is a defensin. In one aspect, the protein is a β-defensin. In a more specific aspect, the protein is a human β-defensin 2.

In a preferred embodiment, the step of transfecting includes transfecting a liposome containing the mRNA into the human cell. Preferably, the human cell is transfected with a concentration of at least about 0.5 μg/ml of the mRNA. In another aspect, the human cell is transfected with a concentration of at least about 2 μg/ml of the mRNA. In yet another aspect, at least about 1 pg of the protein having antimicrobial biological activity is expressed per mg of total cellular protein per μg of nucleic acid transfected into the cell. In another aspect, the transfection efficiency of the method is at least about 50%. In another aspect, the transfection efficiency of the method is at least about 75%. In yet another aspect, the transfection efficiency of the method is at least about 90%. Preferably, the human cell is transfected with an amount of defensin protein that is not toxic to the cell. In one aspect, the human cell expresses the defensin intracellularly. In another aspect, the step of transfecting is performed ex vivo.

Yet another embodiment of the present invention relates to a method for expression of a therapeutic protein in a human primary macrophage. The method includes the step of transfecting the human primary macrophage with a composition comprising:(a) an isolated mRNA encoding a therapeutic protein; and, (b) a liposome delivery vehicle. The isolated mRNA is transfected at a concentration of at least about 0.5 μg/ml mRNA, and the therapeutic protein is expressed by the human primary macrophage.

In one aspect, the mRNA is transfected at a concentration of at least about 1 μg/ml mRNA. In another aspect, the mRNA is transfected at a concentration of at least about 2 μg/ml mRNA. In yet another aspect, the transfection efficiency of the method is at least about 50%. In another aspect, the transfection efficiency of the method is at least about 75%. In another aspect, the transfection efficiency of the method is at least about 90%. In one aspect, at least about 1 pg of the therapeutic protein is expressed per mg of total cellular protein per μg of nucleic acid transfected into the cell.

In a preferred embodiment, the liposome delivery vehicle comprises cationic lipids.

In one aspect, the mRNA encodes a protein that is not naturally expressed by the primary human macrophage. Preferably, the mRNA encodes an antimicrobial protein. Such an antimicrobial protein can include, but is not limited to, a defensin protein. A preferred defensin protein is human β-defensin 2. Preferably, the therapeutic protein is expressed by the human primary macrophage in an amount effective to inhibit growth of a microorganism. Even more preferably, the therapeutic protein is expressed by the human primary macrophage in an amount effective to substantially prevent growth of a microorganism. In one aspect, the step of transfecting is performed ex vivo.

Another embodiment of the present invention relates to a method for treating a disease caused by a pathogenic microorganism in a human patient that is infected by the pathogenic microorganism. The method includes the step of transfecting human primary macrophages in the human patient with a composition comprising: (a) an isolated mRNA encoding a therapeutic protein; and, (b) a liposome delivery vehicle. The isolated mRNA is transfected at a concentration of at least about 0.5 μg/ml mRNA, the therapeutic protein is expressed by the human primary macrophage, and the protein is expressed so that growth of the microorganism is inhibited. In one aspect, the pathogenic microorganism is Mycobacterium tuberculosis, wherein the therapeutic protein is a defensin, and wherein the disease is tuberculosis.

In one aspect, the mRNA encodes an antimicrobial protein. Such an antimicrobial protein can include, but is not limited to, a defensin protein. In one aspect, the mRNA encodes human β-defensin 2. Preferably, the therapeutic protein is expressed by the human primary macrophage in an amount effective to inhibit growth of a microorganism. Even more preferably, therapeutic protein is expressed by the human primary macrophage in an amount effective to substantially prevent growth of a microorganism.

### DETAILED DESCRIPTION OF THE INVENTION

The present invention generally relates to the present inventors' discovery of a highly efficient method for the expression of a therapeutic protein in a human host cell that is naturally resistant to transfection with foreign (i.e., recombinant, derived from an exogenous source) nucleic acids. More particularly, the present inventors have discovered that human primary macrophages, which are normally highly resistant to transfection with nucleic acids, can be successfully transfected with nucleic acids so that effective expression of a therapeutic protein can be achieved. The method includes the transfection of the macrophages with mRNA expressing a therapeutic protein; in a preferred embodiment, the mRNA is complexed with a liposome. The present inventors have demonstrated that not only can primary human macrophages be successfully transfected by this method at very high efficiency which surpasses previously reported transfection efficiency by at least 40-fold, the macrophages can then express the protein in an amount effective to inhibit and even prevent the growth of microorganisms which infect or are otherwise in contact with the cells (i.e., microorganisms that naturally infect the host cells). In some embodiments, the microorganisms are effectively killed by the expression of the antimicrobial according to the present invention. These results are surprising because, prior to the present invention, attempts to transfect human primary macrophages resulted in very poor transfection efficiency, in contrast to the successful transfections achieved in other mammalian cells, including in murine primary macrophages.

As discussed above, although the use of antimicrobial therapeutic proteins such as defensins has been proposed (Ganz and Lehrer, Pharmacology & Therapeutics 66:191-205, 1995), chemical synthesis of these peptides is a challenge due to the complex pattern of disulfide bonds which stabilize the structure (Lauth et al., Insect Biochem Mol Biol 28:1059-66, 1998), and recombinant methods do not produce sufficient yields (Harwig et al., Meth. in Enzymol. 236:160-170, 1994; Valore and Ganz, Methods Mol Biol 78:115-31, 1997). DNA has previously been used to encode defensins for intracellular expression in a murine macrophage cell line, which resulted in greater resistance to Histoplasma capsulatum (Couto et al., Infection & Immunity 62:2375-8, 1994). Additionally, the present inventors have previously observed that primary murine macrophages efficiently accumulate both RNA and DNA delivered as a complex with cationic lipids both in vivo and in vitro (Kisich et al., J Immunol 163:2008-16, 1999; Malone et al., Proc. Natl. Acad. Sci., U.S.A. 86:6077-6081, 1989). However, prior to the present invention, there have been very few reports of primary human macrophage being transfected with DNA plasmids. Moreover, those investigators which have quantitated transfection efficiency report that only about 2% of the cells expressed the transfected gene (i.e., the reporter gene (eGFP)) (Simoes et al., J Leukoc Biol 65:270-9, 1999; Van Tendeloo et al., Gene Ther 5:700-7, 1998; Weir and Meltzer, Cell Immunol 148:157-65, 1993). Therefore, it appeared, prior to the present invention, that human macrophages were not suitable candidates for transfection with exogenous nucleic acids.

In view of the lack of success in transfection of human primary macrophages and expression of exogenous proteins prior to the present invention, it was both unexpected and surprising that the present inventors could transfect human primary macrophages with a nucleic acid that achieved greater than 90% transfection efficiency. As mentioned above, this efficiency is approximately 40-fold greater than has previously been reported for cultured human macrophages using electroporation or lipoplex mediated delivery of DNA reporter vectors (Simoes et al., J Leukoc Biol 65:270-9, 1999; Van Tendeloo et al., Gene Ther 5:700-7, 1998; Weir and Meltzer, Cell Immunol 148:157-65, 1993). Although human macrophages have previously been shown to be very difficult to transfect with plasmid vectors, the present inventors have demonstrated that primary human macrophages can be efficiently transfected with mRNA encoding potentially therapeutic proteins.

The present inventors have also demonstrated that as a result of the highly efficient mRNA transfection described herein, primary human macrophages synthesizing an antimicrobial protein, human β-defensin 2 (hBD-2), displayed mycobactericidal and mycobacteristatic activity. As discussed in detail in the Examples section, immunostaining for hBD-2 after transfection of M. tuberculosis-infected macrophages was mainly observed associated with intracellular M. tuberculosis, rather than in the cytoplasm of the macrophages. Mycobacteria have been reported to reside in phagosomes which do not normally mature to lysosomes (Deretic and Fratti, Mol Microbiol 31:1603-9, 1999). The localization of the expressed hBD-2 to this intracellular compartment was surprising, as it is not obvious how the hBD-2 gained access to the bacilli. In the epithelial cells where hBD-2 is normally synthesized, it is directly secreted via the trans-golgi, and not stored intracellularly (Diamond and Bevins, Clinic. Immunol. and Immunopathol. 88:221-225, 1998). In contrast, the α-defensins are stored in cytoplasmic granules of PMN or paneth cells (Ouellette, Am J Physiol 277:G257-61, 1999 Ouellette, Am J Physiol 277:G257-61, 1999). Without being bound by theory, the present inventors believe that the hBD-2 synthesized from the transfected mRNA was secreted from the macrophages soon after synthesis. After secretion, the newly synthesized hBD-2 would have to gain access to the intracellular bacilli via the endocytic process, or via direct penetration of the macrophage plasma membrane, and then the membrane of the mycobacteria-containing phagosome. The mycobacteria-containing phagosome has also been reported to exchange material with the extracellular medium via the recycling endosome compartment (Clemens and Horwitz, J Exp Med 184:1349-55, 1996). It is therefore possible that hBD-2 secreted by the macrophages re-entered the cells by endocytosis and was then transported into the mycobacteria-containing phagosome. However, as defensins have been shown to bind to and penetrate the plasma membranes of mammalian cells, direct diffusion of the newly synthesized hBD-2 from the trans-golgi or extracellular medium directly into the phagosomes cannot be ruled out. Once exposed to the mycobacterium, the high affinity of defensins for bacterial cell membranes would tend to cause accumulation of the expressed defensin on the surface of the bacilli. This was observed by the present inventors, with immunostaining of hBD-2 mainly localized to the bacilli. The present inventors have also observed high accumulation of fluorescently labeled human neutrophil peptide 1 (HNP-1) on Mycobacterium avium in human macrophages within five minutes of addition to the medium, while similarly labeled bovine serum albumin was excluded from the cells (data not shown).

Therefore, regardless of the exact mechanism, the present inventors also provided evidence that the antimicrobial proteins produced by the human primary macrophages can gain direct access to and contact the target microorganism, which indicates that the produced protein is likely to be able to inhibit the growth of not only the transfected macrophage, but also of neighboring monocytes, epithelial cells, or other host cells which also harbor or contact the target microorganism. Exposure of intracellular mycobacteria to defensins in the extracellular medium helps to explain how alveolar macrophages, which do not normally synthesize defensins, might utilize defensins synthesized by nearby cells, including epithelia and neutrophils to limit multiplication of M. tuberculosis following inhalation and phagocytosis. Therefore, the present inventors have provided a novel method by which the contact of cells with antimicrobials can be enhanced to inhibit the growth of pathogenic microorganisms and thereby inhibit the progression of a disease associated with such a microorganism.

Accordingly, one embodiment of the present invention relates to a method to inhibit the growth of a microorganism. The method includes the step of transfecting a human host cell with an isolated mRNA encoding a protein having antimicrobial activity. The human host cell is characterized by being infected with, susceptible to infection with, or otherwise likely to be in contact with the microorganism or with a cell infected with the microorganism. The human cell expresses the protein encoded by the mRNA, and thereby inhibits the growth of a microorganism at some point after the microorganism contacts and/or infects the human cell or a bystander cell (i.e., a human cell that is not transfected by the mRNA, but which is within the local environment of a transfected cell, such that an antimicrobial protein that is secreted from a nearby transfected cell can come into contact, and potentially enter the bystander cell). The human cell is characterized in that it is a natural host cell for the microorganism (i.e., the cell is a natural host for the microorganism) or naturally comes into contact with the microorganism when a human is infected with the microorganism (i.e., the microorganism interacts or is likely to interact in some physical way with the human host cell during infection of a human with the microorganism). In one embodiment, the human cell does not naturally express the protein (i.e., under normal physiological conditions, the human cell does not express detectable amounts of the protein).

According to the present invention, to inhibit the growth of a microorganism refers to any inhibition (i.e., reduction, lessening, slowing, downregulation, decrease) in the replication (i.e., proliferation or growth) of a microorganism as compared to in the absence of the exposure of the microorganism to an antimicrobial protein according to the present invention. In one embodiment, to inhibit the growth of a microorganism can encompass preventing (i.e., stopping, halting, deterring) the growth of the microorganism (i.e., no detectable growth of the microorganism can be measured), as well as death, or killing, of the microorganism (i.e., the numbers of microbes decreases and indicators of microbe cell death can typically be detected). Typically, the growth of the microorganism after contact with the antimicrobial protein is compared to the growth of the microorganism in the absence of the antimicrobial protein in the culture or cellular milieu under normal in vitro culture conditions or normal physiological conditions (depending on whether the comparison is in vitro or in vivo). Many viruses, bacteria and parasites, for example, replicate in the intracellular compartments of their host cell. Some microorganisms can replicate in the extracellular spaces between cells (e.g., Gram-positive cocci) and evade encapsulation by means of their polysaccharide capsule. The method of the present invention is effective to detectably reduce such replication by both intracellular and extracellular microorganisms, and the present inventors have demonstrated that the method of the present invention can be effective to prevent replication of a microorganism, at least for a period of time. Referring to Example 4, the present inventors have demonstrated that transfection of primary macrophages with a concentration of as little as 0.5 μg/ml of human β-defensin 2 mRNA, but not with a control mRNA, inhibited growth of M. tuberculosis in the macrophages. Growth of M. tuberculosis in the monolayers was prevented by treatment with a concentration of 2 μg/ml (˜20 nM) or more of hBD-2 mRNA.

Preferably, a single administration of mRNA encoding a protein having antimicrobial activity to a human host cell according to the present invention results in at least about a 10% decrease in growth of a microorganism infecting or in contact with the host cell (as compared to in the absence of or prior to transfection of the host cell with the mRNA), over at least about 24 hours, and more preferably, over at least about 2 days (48 hours). More preferably, a single administration of mRNA encoding a protein having antimicrobial activity to a human host cell according to the present invention results in at least about a 20% decrease in growth of a microorganism, and more preferably at least about a 30% decrease, and more preferably at least about a 40% decrease, and more preferably at least about a 50% decrease, and more preferably at least about a 60% decrease, and more preferably at least about a 70% decrease, and more preferably at least about a 80% decrease, and more preferably at least about a 90% decrease, and most preferably at least about a 100% decrease (i.e., prevention of detectable growth), in growth of a microorganism infecting or in contact with the host cell, over at least about 24 hours, and more preferably over at least about 2 days (48 hours). Even more preferably, a single administration of mRNA encoding a protein having antimicrobial activity to a human host cell according to the present invention results in a detectable improvement in the morphology of the host cell, with a statistically significant decrease in the total number of dead cells in a population of host cells (p<0.05) over at least about 2 days. Preferably, a single administration of mRNA encoding a protein having antimicrobial activity to a human host cell according to the present invention results in the above-described inhibition of growth of a microorganism infecting or in contact with the host cell, over at least about 3 days, and more preferably over at least about 4 days, and more preferably over at least about 5 days, and more preferably over at least about 6 days, and even more preferably over at least about 7 days, and even more preferably over at least about 8 days, and even more preferably over at least about 9 days, and even more preferably over at least about 10 days, with even longer periods (i.e., at least about 15, 20, 25, or 30 days) being even more preferred. In another embodiment, a single administration of mRNA encoding a protein having antimicrobial activity to a human host cell according to the present invention, results in the death (i.e., killing) of at least about 5% of the microbes in a given population of microorganisms over the above-referenced time periods. Preferably, at least about 10%, and more preferably at least about 25%, and more preferably at least about 35%, and more preferably at least about 45%, and more preferably at least about 55%, and more preferably at least about 65%, and more preferably at least about 75%, and more preferably at least about 85%, and more preferably at least about 95% of the microbes in a given population of microorganisms are killed over the given time periods.

The growth of a microorganism can be determined by any suitable method for measuring the growth of a microorganism known in the art. For example, as demonstrated in Examples 4 and 5, microorganism growth can be measured by counting the colony forming units (CFU) formed from the culture of a sample of microorganisms, and comparing the CFU before and after a given treatment (i.e., transfection of a cell harboring the microorganism or in contact with the microorganism with the mRNA encoding an antimicrobial protein). Other methods of detecting microorganism growth include, but are not limited to, determining optical density of a culture and microscopy techniques, including immunofluorescent microscopy. Similarly, the death of a microorganism can be measured using methods common in the art, including microscopic techniques, dye exclusion.

As used herein, a human host cell can be any human cell which: (1) can be transfected with an mRNA encoding a protein and express the protein; and, (2) is naturally infected by the microorganism against which the antimicrobial protein is directed, is naturally susceptible to being infected by such microorganism, and/or is otherwise naturally susceptible to being contacted by such microorganism or by a cell infected with such microorganism. As used herein, a cell that is naturally infected by a microorganism or that is naturally susceptible to being infected by a microorganism is a cell that is a naturally occurring host for a pathogenic microorganism. More particularly, such a cell, under normal physiological conditions in vivo or in vitro, can be infected by a microorganism such that the microorganism gains access to the intracellular compartments of the cell (e.g., the cytosol, the endosomes, the lysosomes). Reference to a cell that is infected means that the cell harbors a microorganism (i.e., contains a microorganism intracellularly). A cell that is naturally susceptible to being contacted by a microorganism can include a cell that is susceptible to being infected by a microorganism, since infection requires some initial contact of the cell by the microorganism, but also includes cells that may not be infected by the microorganism, but which may contact the microorganism by chance as a result of being in the local environment of an infection or by being a cell in the preferred area of infection by an extracellular microorganism (e.g., a lung epithelial cell may contact a Streptococcus pneumoniae upon introduction of the bacterium to the lung tissue), or which may contact the microorganism to perform a function of the cell (i.e., a phagocyte contacting a microorganism to phagocytose the microorganism). The term “contact” primarily refers to physical contact of the cell with the microorganism or of the antimicrobial agent produced by the cell with the microorganism (i.e., by secreting an antimicrobial protein, the cell can effectively contact a microorganism). In each of the above-described scenarios, if the cell expresses a protein having antimicrobial activity according to the present invention, the growth of the microorganism can be inhibited by the antimicrobial protein.

Therefore, human cells which are suitable for transfection according to the method of the present invention include, but are not limited to, macrophages, granulocytes or polymorphonuclear leukocytes (PMNs) (e.g., neutrophils, eosinophils, basophils), paneth cells, and epithelial cells. Preferred cells to transfect according to the method of the present invention include macrophages, neutrophils and epithelial cells.

A particularly preferred cell to transfect using the method of the present invention includes a primary human macrophage. Macrophages mature continuously from circulating monocytes and leave the circulation to migrate into tissues throughout the body, where they are found in large numbers in connective tissue and along certain blood vessels in the liver and spleen. These large phagocytic cells play a key part in all phases of host defense. Macrophages in tissues have receptors for various microbial constituents on their surface, as well as Fc receptors and complement receptors, by which they engulf opsonized particles. The microbial constituent receptors include the mannose receptor, the scavenger receptor and receptors for lipopolysaccharide (LPS). When pathogens cross an epithelial barrier they can be recognized by phagocytes such as macrophages, and are trapped, engulfed and destroyed. Some microorganisms, such as Mycobacterium tuberculosis, attempt to evade the phagocytic system, by infecting the macrophage itself, and using the macrophage as a host in which to replicate. A primary macrophage is a macrophage which has been recently differentiated from a monocyte, and typically which have not yet begun to display characteristics of more mature macrophages which are resident in different tissues. In one embodiment, the primary human macrophage is preferably from lung tissue (i.e., under normal physiological conditions, can be isolated from lung tissue). Methods for producing primary macrophages in vitro are exemplified in Example 1.

The method of the present invention is useful for inhibiting the growth of a microorganism. Therefore, it will be clear to those of skill in the art that it is preferred to inhibit the growth of a pathogenic microorganism, in order to reduce the symptoms and tissue damage that are frequently associated with infection by a pathogenic microorganism. However, it will be appreciated that there can also be scenarios in which it is desirable to inhibit the growth of a microorganism that is not necessarily considered to be pathogenic. For example, the normal microbial flora that is characteristic of many regions of the body (e.g., the gastrointestinal tract, the reproductive tract in females) is typically beneficial to the human host. Such microorganisms are frequently referred to as “beneficial” microorganisms. However, the natural balance of a particular beneficial microorganism relative to others can occasionally become skewed (e.g.., due to a physiological change in the human host) such that the human host experiences discomfort, pain, tissue damage, and/or other problems or as a result of the overgrowth of the microorganism. In this scenario, it is desirable to inhibit the growth of the normally “beneficial” microorganism to return the tissue to the normal microbial balance.

Accordingly, the method of the present invention can be used to inhibit both pathogenic and non-pathogenic microorganisms, with the inhibition of the growth of pathogenic microorganisms being particularly preferred. As used herein, a “pathogenic microorganism” is any microorganism that causes a pathology (e.g., damage, infectious disease) in a human or other animal. Such microorganisms enter characteristic sites in the body where they produce disease by a variety of mechanisms. Infection by such a microorganism usually leads to a perceptible disease, where the infected animal can experience discomfort, distress, inflammation, pain, and tissue damage, among other possible symptoms. Pathogenic microorganisms can be extracellular (i.e., replicate in the extracellular spaces between cells) or intracellular (i.e., replicate in an intracellular compartment), and cause tissue damage to a host organism by both direct and indirect methods. Direct methods include: exotoxin production, endotoxin production and direct cytopathic effects. Indirect methods include: elicitation of immune complexes, elicitation of anti-host antibody, and induction of cell-mediated immunity, such mechanisms having a damaging effect on the host tissues in the effort to eradicate the microorganism.

Pathogenic microorganisms which can be inhibited by the present method include, but are not limited to, a bacterium, a fungus, a virus, a protozoa and a parasite, wherein the given microorganism is considered by those in the art to be pathogenic, as discussed above.

Preferred bacteria to inhibit include both Gram-positive and Gram-negative bacteria such as, but not limited to: Gram (+) cocci (e.g., Staphylococci, Streptococci), Gram (−) cocci (e.g., Neisseriae), Gram (+) bacillus (e.g., Bacillus, Listeria), Gram (−) bacillus (e.g., Salmonella, Shigella, Vibrio, Yersinia, Legionella, Bordetella, Pseudomonas), anaerobic bacteria (e.g., Clostridia), spirochetes, mycobacteria (e.g., M. tuberculosis, M. avium, M. leprae), rickettsias, Chlamydias and mycoplasmas. Particularly preferred bacteria to inhibit using the method of the present invention include mycobacteria, with inhibition of the growth of Mycobacterium tuberculosis being preferred in one embodiment.

Preferred fungi of which to inhibit the growth by the method of the present invention include: pathogenic yeast, molds and dimorphic fungi. Particularly preferred fungi include, but are not limited to: Candida albicans, Cryptococcus neoformans, Aspergillus, Histoplasma capsulatum, Coccidioides iminitis, and Pneumocystus carinii.

Preferred viruses of which to inhibit the growth by the method of the present invention include, but are not limited to, enveloped viruses, including, but not limited to, Herpesviruses (e.g., herpes simplex virus), Hepadnaviruses (e.g., Hepatitis B), and human immunodeficiency viruses.

Preferred protozoa of which to inhibit the growth by the method of the present invention include, but are not limited to: Giardia, Leishmania, Plasmodium, Trypanosoma, and Toxoplasma.

Preferred parasites of which to inhibit the growth by the method of the present invention include, but are not limited to: Trichinella, Ascaris, Filaria, Onchocerca, and Schistosoma.

According to the present invention, the mRNA encodes a protein having antimicrobial activity (also referred to herein as an antimicrobial protein). As used herein, “antimicrobial activity” is defined as any activity of a protein which has the general characteristic of being able to reduce the growth of, damage, and/or neutralize the activity of the microorganism. More specifically, a protein with antimicrobial activity is any protein (including peptides) which inhibits or destroys a microbe by depriving it of essential nutrients, such as iron, or by causing structural disruption or metabolic injury to the microorganism. Antimicrobial agents are described in detail in Martin et al., 1995, J. Leuk. Biol. 58:128-136; and Diamond et al., 1998, Clin. Immunol. Pathol. 88:221-225, both of which are incorporated herein by reference, and much of the discussion of antimicrobial agents below, as well as the table below, can be found in these references. All antimicrobial agents discussed in Martin et al., ibid. or Diamond et al., ibid., or otherwise known in the art can be used in the present invention, as well as variants of such antimicrobial agents, similar antimicrobial proteins, and peptide mimetics thereof.

The initial interaction between pathogenic microbes and higher eukaryotes usually takes place at an epithelial surface where microbes adhere, and, if they survive, either multiply locally or penetrate into deeper tissue layers. Host-derived antimicrobial substances released at sites of microbial invasion range in complexity from relatively simple inorganic molecules, such as hydrogen peroxide, nitric oxide, or hypochlorous acid, to antimicrobial peptides, proteins, and multimeric protein complexes, such as complement. The present invention encompasses the production of any antimicrobial proteins and peptides (e.g., <100 amino acids) which can be encoded by mRNA and which can be expressed in a human host cell according to the present invention.

Although the antimicrobial peptides are impressively diverse in structure, most are cationic (positively charged) and amphiphilic. These features facilitate interaction with negatively charged microbial surface structures. Almost all of the peptides investigated in detail damage by first binding and then inserting into the microbial lipid membrane, thereby altering membrane permeability and impairing internal homoeostasis. An affinity for acidic cell wall and membrane constituents (e.g., teichoic acids and phospholipids) may contribute to target cell specificity whereas the conformational structure (amphiphilic β sheet, amphiphilic α-helix, or linear) may dictate the mode of insertion into membranes. In contrast to most of the conventional antibiotics in bacteria and fungi, which are produced by complex metabolic pathways, the antimicrobial peptides of higher eukaryotes are products of single genes and are expressed in specialized cells. They are either stored in specific subcellular compartments and delivered on stimulation or their synthesis and release is triggered by microbes or microbial products, such as lipopolysaccharide.

TABLE 1
Mammalian and Avian Disulfide-Linked Antimicrobial Molecules

| Subfamily | Name | Abbreviation | Synonym | Animal | Localization | References |
|---|---|---|---|---|---|---|
| Classical defensins | Human neutrophil | HNP 1-4 | Corticostatin (CS) | Human | Neutrophil | 1, 2, 3 |
| | peptide | | | | | |
| | Human defensin | HD 5, 6 | | Human | Paneth cell | 1, 2, 3 |
| | Neutrophil peptide | NP 1-6 | Corticostatin (CS) | Rabbit | Neutrophil | 1, 2, 3 |
| | Macrophage cationic | MCP-1, -2 | NP 1, 2 | Rabbit | Alveolar | 1, 2, 3 |
| | peptide | | | | macrophage | |
| | Cryptdin | CRYPT 1-20 | CRYPTA = CRYPT 1 | Mouse | Paneth cell | 1, 2, 3, 4, 5 |
| | Rat neutrophil peptide | RatNP 1-4 | RtNP 1-4 | Rat | Neutrophil | 1, 2, 3 |
| | | | RTNP 1-4 | | | |
| | | | Rat corticostatin (R1-5) | | | |
| | Guinea pig (gp) | GPDEF-1, -2 | Guinea pig neutrophil | Guinea pig | Neutrophil | 1, 2, 3 |
| | defensin | | peptide (GNP or GPNP), | | | |
| | | | guinea pig neutrophil | | | |
| | | | cationic peptide (GNCP), | | | |
| | | | and guinea pig | | | |
| | | | corticostatin (GPCS 1-3) | | | |
| β-Defensins | Tracheal antimicrobial | TAP | | Bovine | Columnar epithelial | 6, 7, 8 |
| | peptide | | | | cells in the upper | |
| | | | | | respiratory tract | |
| | Lingual antimicrobial | LAP | | Bovine | Tongue epithelium | 9 |
| | peptide | | | | | |
| | Bovine neutrophil beta | BNBD 1-13 | | Bovine | Neutrophil | 10, 11 |
| | defensin | | | | | |
| | Gallinacin | GAL 1-3 | CHP -1, -2 (chicken | Chicken | Heterophil | 12, 13, 14 |
| | | | heterophil peptide -1, -2 | | (neutrophil | |
| | | | | | equivalent) | |
| | Turkey heterophil | THP 1-3 | | Turkey | Heterophil | 14 |
| | peptide | | | | (neutrophil | |
| | | | | | equivalent) | |
| Disulfide-linked β- | Protegrin | PG 1-4 | | Pig | Leukocyte^{a} | 15, 16, 17 |
| sheet peptide other | | | | | | |
| than defensin | | | | | | |
| Cysteine disulfide | Cyclic dodecapeptide | bac | Bactenecin | Bovine | Neutrophil | 18, 19, 20 |
| ring peptide | | | | | | |

^{a}Not yet further specified.
References:
1. Lehrer et al., Annu. Rev. Immunol., 11:105-128, 1993
2. Ganz and Lehrer, Curr. Opin. Immunol., 6:584-589, 1994 (abstract)
3. Kagan et al., Toxicology, 87:131-149, 1994
4. Aley et al., Infect. Immun., 62:5397-5403, 1994
5. Huttner et al, Genomics, 19:448-453, 1994
6. Diamond et al., Proc. Natl. Acad. Sci. USA, 88:3952-3956, 1991
7. Diamond et al., Proc. Natl. Acad. Sci. USA, 90:4596-4600, 1993
8. Diamond and Bevins, Chest, 105:51S-52S, 1994 (abstract)
9. Barry et al., Science, 267:1645-1648, 1995
10. Selsted et al., J. Biol. Chem., 268:6641-6648, 1993
11. Tang and Selsted, J. Biol. Chem., 268:6649-6653, 1993
12. Harwig et al., FEBS Lett., 342:281-285, 1994
13. Harwig et al., Techniques in Protein Chemistry V (J. W. Crabb, ed.), Academic Press, San Diego, CA, 81-88, 1994
14. Evans et al., J. Leukoc. Biol., 56:661-665. 1994
15. Kokryakov et al., FEBS Lett., 327:231-236, 1993
16. Storici and Zanetti, Biochem. Biophys. Res. Commun., 196:1363-1368, 1993
17. Zhao et al., FEBS Lett., 346, 285-288, 1994
18. Romeo et al., J. Biol. Chem., 263:9573-9575, 1988
19. Schluesener et al., J. Neuroimmunol., 47:199-202, 1993
20. Storici et al., FEBS Lett., 314:187-190, 1992

A preferred protein having antimicrobial activity for use in the present invention is a defensin protein. As set forth above, the defensins are a broad class of cationic peptides that are found in a variety of mammalian myeloid and epithelial cells, and are bactericidal or bacteristatic for a broad spectrum of microbes. Classical defensins (Lehrer et al., Annu. Rev. Immunol., 11:105-128, 1993) are active against bacteria (Gram positive and Gram negative, including spirochetes and mycobacteria), fungi (yeasts, molds, and dimorphic), and certain enveloped viruses (including herpes simplex virus and human immunodeficiency virus). Most recently the mouse intestinal defensin cryptdin has been shown to be active against the protzoa Giardia lamblia (Aley et al., Infect. Immun., 62:5397-5403, 1994). A similarly broad spectrum has been found for protegrins (Kokryakov et al., FEBS Lett., 327:231-236, 1993). β-Defensins are active against bacteria (Gram positive and Gram negative) and fungi (Diamond et al., Proc. Natl. Acad. Sci. U.S.A., 88:3952-3956, 1991; Selsted et al., J. Biol. Chem., 268:6641-6648, 1993; Harwig et al., FEBS Lett., 342:281-285, 1994; Harwig et al., Techniques in Protein Chemistry V (J. W. Crabb, ed.), Academic Press, San Diego, Calif., 81-88, 1994), whereas the bovine leukocyte cyclic dodecapeptide only shows activity against gram-positive and -negative bacteria (Romeo et al., J. Biol. Chem., 263:9573-9575, 1988). The linear bovine peptides Bac5 and Bac7 are active against Gram-negative bacteria (Skerlavaj et al., Infect. Immun., 58:3724-3730, 1990; Scocchi et al., Eur. J. Biochem., 209:589-595, 1992), including spirochetes (Scocchi et al., Infect. Immun., 61:3081-3083, 1993 (abstract)), and the bovine peptide indolicidin was found to be active against Gram-positive and Gram-negative bacteria (Selsted et al., J. Biol. Chem., 267:4292-4295, 1992). The pig intestinal cecropin, P1, like the insect cecropins, is active against Gram-negative bacteria (Lee et al., Proc. Natl. Acad. Sci. U.S.A., 86:9159-9162, 1989) and porcine PR39 kills Gram-negative and Gram-positive bacteria (Agerberth et al., Eur. J. Biochem., 202:849-854, 1991).

Overall, the microbicidal concentrations of these peptides range between 1 and 100 μg/ml in the absence of serum and are greatly effected by the test system applied. Conditions of microbicidal activity have been elaborated primarily for classical defensins (Lehrer et al., Infect. Immun., 49:207-211, 1985; Lehrer et al., J. Clin. Invest., 81:1829-1835, 1988; Lehrer et al., J. Clin. Invest., 84:553-561,- 1989). In general, their bactericidal activity increases in proportion to their net positive charge. In the presence of salt and divalent cations (millimolar concentrations of Ca²⁺ or Mg²⁺) activity is substantially diminished against Gram-negative organisms and Candida albicans but is retained against Gram-positive bacteria, filamentous fungi, and viruses. Salts and divalent cations also diminish the activity of Bac5 and Bac7, whereas lactoferrin greatly potentiates their activity (Skerlavaj et al., Infect. Immun., 58:3724-3730, 1990). Classical defensins are considerably less active in the presence of serum, as a consequence of their binding by α₂-macroglobulin (Panyutich and Ganz, Am. J Respir. Cell. Mol. Biol., 5:101 -106,1991), certain complement components (Panyutich et al., FEBS Lett., 356:169-173, 1994), and other serum proteins (Panyutich et al., Am. J. Respir. Cell. Mol. Biol., 12:351-357, 1995). In contrast, protegrins maintain full activity in the presence of serum (R. I. Lehrer et al., unpublished data). The membrane composition of the microbial target, its metabolic phase, and the expression of certain virulence genes, e.g., the pho P locus of pathogenic Salmonella typhimurium strains, substantially affect the microbe's sensitivity to defensins (Fields et al., Science, 243:1059-1062, 1989; Miller et al., Infect. Immun., 58:3706-3710, 1990; Miller, Mol. Microbiol., 5:2073-2078, 1991; Fujii et al., Protein Sci., 2:1301-1312, 1993).

A particularly preferred defensin for use in the present invention is a β-defensin and of the β-defensins, human β-defensin 2 (hBD-2) is preferred. The β-defensins are a recently discovered class of defensins which are widely distributed in epithelial tissues and leukocytes of birds and mammals. In humans, an abundant β-defensin peptide (hBD1) was initially discovered by analysis of large quantities of hemofiltrate (Bensch et al., FEBS Lett., 368:331-335,1995). Subsequently Ganz and colleagues have isolated hBD-1 from urine and cervical mucous, suggesting that this peptide plays an antimicrobial role in the genitourinary tract (Valore et al., J. Clin. Invest., 101: 1633-1642, 1998). A second β-defensin, hBD2, was identified in psoriatic skin, produced by keratinocytes, suggesting that β-defensins contribute to the expansive surface of the integument (Harder et al., Nature, 387:861, 1997). Nucleic acid sequences (genes and cDNA) encoding β-defensins have also been identified in mouse (Morrison et al., Mamm. Genome, 9:453-457, 1998; Huttner et al., FEBS Lett., 413:45-49, 1997; Bals et al., Infect. Immun., 66:1225-1232,1998), pig (Zhang et al., FEBS Lett., 424:37-40, 1998), and sheep (Huttner et al., J. Nutr., 128:297S-299S, 1998), with expression patterns similar to those of other β-defensins in the human and cow.

Several studies have suggested a role for β-defensins in host defense against infections. In cattle, increased expression of β-defensins is induced near sites of injury and/or inflammation. Three examples include increased β-defensin expressions in bronchioles of Pasteurella-infected lung tissue (Stolzenberg et al., Proc. Natl. Acad. Sci. U.S.A., 94:8686-8690, 1997), increased EBD expression in intestinal epithelial cells of calves infected with Cryptospiridium parvum (Tarver al., Infect. Immun., 66:1045-1056, 1998), and increased LAP expression in tongue epithelial cells adjacent to inflamed grazing wounds (Schonwetter et al., Science, 267:1645-1648, 1995).

The cDNAs of representative members of the major antimicrobial peptide families have been sequenced (Diamond et al., Proc. Natl. Acad. Sci. U.S.A., 88:3952-3956, 1991; Daher al., Proc. Natl. Acad. Sci. U.S.A., 85:7327-7331, 1988; Ouellette et al., J. Cell Biol., 108:1687-1695, 1989; Nagaoka et al., FEBS Lett., 280:287-291, 1991; Del Sal et al., Biochem. Biophys. Res. Commun., 187:467-472, 1992; Jones and Bevins, J. Biol. Chem., 267:23216-23225, 1992; Storici et al., FEBS Lett., 314:187-190, 1992; Jones and Bevins, FEBS Lett.,315:187-192,1993; Palfree et al., Mol. Endocrinol., 7:199-205, 1993; Storici and Zanetti, Biochem. Biophys. Res. Commun., 196:1058-1065, 1993; Storici and Zanetti, Biochem. Biophys. Res. Commun., 196:1363-1368, 1993; Zanetti et al., J. Biol. Chem., 268:522-526, 1993). They are initially translated as preproproteins that contain a signal sequence (prepiece) for targeting to the endoplasmic reticulum and additional proregion(s) not found in the mature peptides. Postranslational proteolytic processing is required to convert these precursor peptides to their mature forms.

The genes of several human (Jones and Bevins, J. Biol. Chem., 267:23216-23225, 1992; Jones and Bevins, FEBS Lett., 315:187-192, 1993; Palfree et al., Mol. Endocrinol., 7:199-205, 1993; Lanzmeier et al., FEBS Lett., 321:267-273, 1993), rabbit (Ganz et al., J. Immunol., 143:1358-1365, 1989), mouse (Ouellette and Lualdi, J. Biol. Chem., 265:9831-9837, 1990; Lin et al., Genomics, 14:363-368, 1992; Huttner et al., Genomics, 19:448-453, 1994), and guinea pig (Nagaoka et al., FEBS Lett., 303:31-35, 1992; Nagaoka et al., Comp. Biochem. Physiol., 106:387-390, 1993; Nagaoka et al., DNA Sequence, 4:123-128, 1993) classical defensins and the bovine β-defensin tracheal antimicrobial peptide (Diamond et al., Proc. Natl. Acad. Sci. U.S.A., 90:4596-4600, 1993) have been cloned and sequenced.

According to the method of the present invention, a human host cell is transfected with mRNA encoding a protein having antimicrobial activity as described above. The mRNA includes the nucleic acid sequence encoding the protein to be expressed (i.e., the coding region), and typically comprises a poly-A tail at the 3′ terminus. Methods for producing mRNA encoding a given protein are known in the art and include in vitro transcription of an mRNA sequence from a DNA sequence (e.g., a cDNA sequence encoding a desired protein). Briefly, a DNA fragment comprising the coding sequence of a desired protein can be isolated and amplified, if necessary. Preferably, capped mRNA encoding the desired protein is made using any in vitro transcription method. Any remaining DNA template is removed and the mRNA is preferably purified by any suitable method for purification of mRNA (e.g., phenol:chloroform extraction) and/or filtration centrifugation. The resulting mRNA preferably has a A₂₆₀/A₂₈₀ ratio of at least about 1.8, and more preferably at least about 1.85, and more preferably at least about 1.9 and even more preferably at least about 1.95, with a A₂₆₀/A₂₈₀ ratio of 2.0 representing theoretically pure mRNA. Kits for performing in vitro transcription are commercially available (e.g., Message Machine kit (Ambion, Austin Tex.)) and the use of such a kit is described in Example 1.

The mRNA is transfected into the host cell in an amount that achieves expression of the antimicrobial protein that is effective to inhibit the growth of a microorganism, and in an amount that is not toxic to the host cell. Preferably, the mRNA is transfected into the host cell in an amount that, in a single administration, achieves expression of the antimicrobial protein effective to inhibit the growth of a microorganism infecting or in contact with the host cell by at least about 10% as compared to in the absence of or prior to transfection of the host cell with the mRNA, over at least about 24 hours and more preferably, over at least about 2 days (48 hours). More preferably, the mRNA is transfected into the host cell in an amount that, in a single administration, achieves expression of the antimicrobial protein effective to inhibit the growth of a microorganism infecting or in contact with the host cell by at least about 20%, and more preferably at least about 30%, and more preferably at least about 40%, and more preferably at least about 50%, and more preferably at least about 60%, and more preferably at least about 70%, and more preferably at least about 80%, and more preferably at least about 90%, and more preferably at least about 100%, as compared to in the absence of or prior to transfection of the host cell with the mRNA, over at least about 24 hours, and more preferably over at least about 2 days (48 hours). More preferably, the mRNA is transfected into the host cell in an amount that, in a single administration, achieves expression of the antimicrobial protein effective to inhibit the growth of a microorganism infecting or in contact with the host cell by any of the above percentages, as compared to in the absence of or prior to transfection of the host cell with the mRNA, over at least about 3 days, and more preferably over at least about 4 days, and more preferably over at least about 5 days, and more preferably over at least about 6 days, and even more preferably over at least about 7 days, and even more preferably over at least about 8 days, and even more preferably over at least about 9 days, and even more preferably over at least about 10 days, with even longer periods (i.e., at least about 15, 20, 25, or 30 days) being even more preferred.

In one embodiment, the mRNA is transfected into the host cell at a concentration of at least about 0.1 μg/ml, and more preferably at least about 0.2 μg/ml, and more preferably at least about 0.5 μg/ml , and more preferably at least about 1 μg/ml , and more preferably at least about 2 μg/ml, and more preferably at least about 3 μg/ml, and more preferably at least about 4 μg/ml , and more preferably at least about 5 μg/ml, and more preferably at least about 6 μg/ml , and more preferably at least about 7 μg/ml, and more preferably at least about 8 μg/ml. Amounts greater than 8 μg/ml can be used provided that such amounts do not result in production of an amount of antimicrobial protein that is toxic to the host cell. Determination of toxic amounts is within the ability of those of skill in the art and is exemplified in the Examples section below with regard to two proteins. It is noted that many of these concentrations are exemplified in the Examples section and that a concentration of mRNA of about 2 μg/ml was calculated to represent about 20 nM mRNA.

In another embodiment, the mRNA is transfected into the cell in an amount effective to achieve production of at least about 1 picogram (pg) of protein expressed per milligram (mg) of total cellular protein per microgram (μg) of nucleic acid delivered. More preferably, the mRNA is transfected into the cell in an amount effective to achieve production of at least about 10 pg of protein expressed per mg of total cellular protein per μg of nucleic acid delivered; and even more preferably, at least about 50 pg of protein expressed per mg of total cellular protein per μg of nucleic acid delivered; and most preferably, at least about 100 pg of protein expressed per mg of total cellular protein per μg of nucleic acid delivered.

In yet another embodiment, the mRNA is transfected into the human host cell with a transfection efficiency of at least about 25% (i.e., 25% of the total number of host cells contacted with the mRNA are successfully transfected and express the antimicrobial protein). Preferably, the mRNA is transfected into the human host cell with a transfection efficiency of at least about 40%, and more preferably at least about 50%, and more preferably at least about 60%, and more preferably at least about 70%, and more preferably at least about 75%, and more preferably at least about 80%, and more preferably at least about 90%, and even more preferably at least about 95%. The present inventors have demonstrated a transfection efficiency in primary human macrophages of greater than 90% using the present method, which is at least 40-fold greater efficiency than previously reported transfection efficiencies for primary human macrophages.

The mRNA encoding the protein having antimicrobial activity is transfected into a human host cell using any suitable method for transfection of an mRNA into such a cell (i.e., transfection, electroporation, microinjection, lipofection, adsorption, viral infection, naked DNA injection and protoplast fusion). The present inventors have found that particularly high efficiency transfection of mRNA into a host cell, and particularly into the transfection-resistant primary human macrophages, can be achieved by complexing the mRNA with a liposome, wherein the complex is then used to transfect the human cell. Therefore, a particularly preferred method of transfecting a human cell, and particularly a primary human macrophage, is by liposome delivery of the mRNA into the cell (i.e., lipofection). A liposome that is complexed with mRNA and used to deliver the mRNA into the cell according to the present invention can also be referred to herein as a liposome delivery vehicle.

A liposome delivery vehicle of the present invention comprises a lipid composition that is capable of fusing with the plasma membrane of the target cell to deliver the recombinant nucleic acid molecule into a cell. Suitable liposomes for use with the present invention include any liposome. Preferred liposomes of the present invention include those liposomes commonly used in, for example, gene delivery methods or in vitro transfection methods known to those of skill in the art. Preferred liposome delivery vehicles comprise multilamellar vesicle (MLV) lipids and extruded lipids. Methods for preparation of MLV's are well known in the art and are described, for example, in the Examples section. According to the present invention, “extruded lipids” are lipids which are prepared similarly to MLV lipids, but which are subsequently extruded through filters of decreasing size, as described in Templeton et al., 1997, Nature Biotech., 15:647-652, which is incorporated herein by reference in its entirety. Small unilamellar vesicle (SUV) lipids can also be used in the composition and method of the present invention. In a particularly preferred embodiment, liposome delivery vehicles comprise liposomes having a polycationic lipid composition (i.e., cationic liposomes) and/or liposomes having a cholesterol backbone conjugated to polyethylene glycol. In a preferred embodiment, liposome delivery vehicles useful in the present invention comprise one or more lipids selected from the group of DOTMA, DOTAP, DOTIM, DDAB, and cholesterol. As noted in the Examples, a particularly preferred liposome for use in the present invention is the composition of lipids represented by Oligofectin G (Sequitur, Natik MA).

A liposome delivery vehicle of the present invention can be modified to target a particular site in a mammal (i.e., a targeting liposome), thereby targeting and making use of a nucleic acid molecule of the present invention at that site. Suitable modifications include manipulating the chemical formula of the lipid portion of the delivery vehicle. Manipulating the chemical formula of the lipid portion of the delivery vehicle can elicit the extracellular or intracellular targeting of the delivery vehicle. For example, a chemical can be added to the lipid formula of a liposome that alters the charge of the lipid bilayer of the liposome so that the liposome fuses with particular cells having particular charge characteristics. Other targeting mechanisms include targeting a site by addition of exogenous targeting molecules (i.e., targeting agents) to a liposome (e.g., antibodies, soluble receptors or ligands). As used herein, the term “target cell” or “targeted cell” refers to a cell to which an mRNA of the present invention is selectively designed to be delivered. The term target cell does not necessarily restrict the delivery of the mRNA only to the target cell and no other cell, but indicates that the delivery of the mRNA, the expression of the mRNA, or both, are specifically directed to a preselected host cell. Targeting liposome delivery vehicles are known in the art. For example, a liposome can be directed to a particular target cell or tissue by using a targeting agent, such as an antibody, soluble receptor or ligand, incorporated with the liposome, to target a particular cell or tissue to which the targeting molecule can bind. Targeting liposomes are described, for example, in Ho et al., 1986, Biochemistry 25: 5500-6; Ho et al., 1987a, J Biol Chem 262: 13979-84; Ho et al., 1987b, J Biol Chem 262: 13973-8; and U.S. Pat. No. 4,957,735 to Huang et al., each of which is incorporated herein by reference in its entirety).

A liposome delivery vehicle is preferably capable of remaining stable in culture (i.e., in vitro) or in a host organism, when delivered in vivo, for a sufficient amount of time to deliver the mRNA into the host cell that is to be transfected with the mRNA. Preferably, a liposome delivery vehicle is stable in culture or in the host organism for at least about 30 minutes, more preferably for at least about 1 hour and even more preferably for at least about 24 hours. A preferred liposome delivery vehicle of the present invention is from about 0.01 microns to about 1 microns in size.

Complexing a liposome with an mRNA of the present invention can be achieved using methods standard in the art and is demonstrated, for example, in the Examples section below. A suitable concentration of an mRNA of the present invention to add to a liposome includes a concentration effective for delivering a sufficient amount of mRNA into a host cell such that the antimicrobial protein encoded by the mRNA can be expressed in an amount effective to inhibit the growth of a microorganism that infects or otherwise contacts the host cell. Preferred amounts of mRNA to transfect have been discussed in detail above. Preferably, from about 0.1 μg to about 10 μg of mRNA of the present invention is combined with about 0.2 nmol to about 20 nmol liposomes. In one embodiment, the ratio of nucleic acids to lipids (μg nucleic acid:nmol lipids) in a composition of the present invention is preferably at least from about 1:10 to about 10:1 nucleic acid:lipid by weight (i.e., 1:10=1 μg nucleic acid:10 nmol lipid), and more preferably at least about from 1:2 to about 6:1 nucleic acid:lipid by weight (i.e., 1:2=1 μg nucleic acid:2 nmol lipid). Preferably, the ratio of nucleic acids to lipids in a composition of the present invention is from about 1:1 to 4:1, and more preferably, 2:1. Other optimum ratios are described in detail in the Examples section.

In one embodiment, a composition of the present invention comprising mRNA and a liposome delivery vehicle can further comprise a pharmaceutically acceptable excipient. As used herein, a pharmaceutically acceptable excipient refers to any substance suitable for delivering a composition useful in the method of the present invention to a suitable ill vivo, ex vivo or in vitro site. Preferred pharmaceutically acceptable excipients are capable of maintaining a nucleic acid molecule of the present invention in a form that, upon arrival of the nucleic acid molecule to a cell, the nucleic acid molecule is capable of entering the cell and being expressed by the cell. Suitable excipients of the present invention include excipients or formularies that transport, but do not specifically target a nucleic acid molecule to a cell (also referred to herein as non-targeting carriers). Examples of pharmaceutically acceptable excipients include, but are not limited to water, phosphate buffered saline, Ringer's solution, dextrose solution, serum-containing solutions, Hank's solution, other aqueous physiologically balanced solutions, oils, esters and glycols. Aqueous carriers can contain suitable auxiliary substances required to approximate the physiological conditions of the recipient, for example, by enhancing chemical stability and isotonicity. Suitable auxiliary substances include, for example, sodium acetate, sodium chloride, sodium lactate, potassium chloride, calcium chloride, and other substances used to produce phosphate buffer, Tris buffer, and bicarbonate buffer. Auxiliary substances can also include preservatives, such as thimerosal, m- or o-cresol, formalin and benzol alcohol.

Proteins produced by the methods of the present invention may either remain within the human host cell; be secreted into the extracellular milieu; be secreted into a space between two cellular membranes; or be retained on the outer surface of a cell or microorganism. In one embodiment, the human cell expresses said protein intracellularly. Preferably, the protein is secreted by the cell so that the antimicrobial protein can enter or attach to extracellular microorganisms or to neighboring (bystander) cells which may be infected by, susceptible to infection by, or otherwise come into contact with, a microorganism of which growth is to be inhibited.

According to the present invention, suitable methods of administering a composition comprising an mRNA encoding an antimicrobial protein of the present invention to human host cell include any route of in vivo administration that is suitable for delivering a recombinant nucleic acid molecule into a patient. The preferred routes of administration will be apparent to those of skill in the art, depending on the type of delivery vehicle used, the target cell population, and the disease or condition experienced by the patient. According to the present invention, the composition comprising the mRNA encoding a protein to be delivered to a human host cell can be delivered to the host cell by any in vivo, ex vivo or in vitro method which results in transfection of the desired host cell with the mRNA. Preferred methods of in vivo administration include, but are not limited to, intravenous administration, intraperitoneal administration, intramuscular administration, intracoronary administration, intraarterial administration (e.g., into a carotid artery), subcutaneous administration, transdermal delivery, intratracheal administration, subcutaneous administration, intraarticular administration, intraventricular administration, inhalation (e.g., aerosol), intracerebral, nasal, oral, pulmonary administration, impregnation of a catheter, and direct injection into a tissue.

Ex vivo refers to performing part of the regulatory step outside of a host organism, such as by transfecting a population of cells removed from a patient with an mRNA comprising a nucleic acid sequence encoding an antimicrobial protein according to the present invention under conditions such that the mRNA is subsequently expressed by the transfected cell, and returning the transfected cells to the-host organism. Methods to achieve such transfection include, but are not limited to, transfection, viral infection, electroporation, lipofection, bacterial transfer, spheroplast fusion, and adsorption. As discussed above, the mRNA is preferably complexed with a liposome delivery vehicle for transfection into the host cell. Ex vivo methods are particularly suitable when the target cell can easily be removed from and returned to the host organism.

Intravenous, intraperitoneal, and intramuscular administrations can be performed using methods standard in the art. Aerosol (inhalation) delivery can also be performed using methods standard in the art (see, for example, Stribling et al., Proc. Natl. Acad. Sci. U.S.A. 189:11277-11281, 1992, which is incorporated herein by reference in its entirety). Oral delivery can be performed by complexing a therapeutic composition of the present invention to a carrier capable of withstanding degradation by digestive enzymes in the gut of an animal. Examples of such carriers, include plastic capsules or tablets, such as those known in the art.

One method of local administration is by direct injection. Direct injection techniques are particularly useful for administering an mRNA to a cell or tissue that is accessible by surgery, and particularly, on or near the surface of the body. Administration of a composition locally within the area of a target cell refers to injecting the composition centimeters and preferably, millimeters from the target cell or tissue.

Various methods of administration and delivery vehicles disclosed herein have been shown to be effective for delivery of a nucleic acid molecule to a target cell, whereby the nucleic acid molecule transfected the cell and was expressed. In many studies, successful delivery and expression of a heterologous gene was achieved in preferred cell types and/or using preferred delivery vehicles and routes of administration of the present invention. All of the publications discussed below and elsewhere herein with regard to gene delivery and delivery vehicles are incorporated herein by reference in their entirety. For example, using liposome delivery, U.S. Pat. No. 5,705,151, issued Jan. 6, 1998, to Dow et al. demonstrated the successful in vivo intravenous delivery of a nucleic acid molecule encoding a superantigen and a nucleic acid molecule encoding a cytokine in a cationic liposome delivery vehicle, whereby the encoded proteins were expressed in tissues of the animal, and particularly in pulmonary tissues. As discussed above, Liu et al., 1997, ibid. demonstrated that intravenous delivery of cholesterol-containing cationic liposomes containing genes preferentially targets pulmonary tissues and effectively mediates transfer and expression of the genes in vivo. PCT Publication No. WO99/66879 to Dow et al. further demonstrates the successful in vivo administration of polyA-enriched RNA from tumor cells complexed to a cationic lipid in mice. Examples 1-5 below further demonstrate the successful delivery and expression of an mRNA of the present invention in vitro.

Yet another embodiment of the present invention relates to a method for expression of a protein in a human primary macrophage. Preferably, the method is used to express a therapeutic protein in a human primary macrophage. The method includes the step of transfecting the human primary macrophage with a composition comprising: (a) an isolated mRNA encoding a therapeutic protein; and, (b) a liposome delivery vehicle. The isolated mRNA is transfected at a concentration of at least about 0.5 μg mRNA per ml of liposome such that the therapeutic protein is expressed by the human primary macrophage. Preferably, the therapeutic protein is a protein that is not naturally expressed by the primary human macrophage.

According to the present invention, a “therapeutic protein” is any protein from which a therapeutic benefit can be derived. The therapeutic benefit can be any measurable, observable or perceived benefit from the protein for any animal, and preferably humans. Therefore, a therapeutic benefit is not necessarily a cure for a particular disease or condition, but rather, preferably encompasses a result which can include alleviation of the disease or condition, elimination of the disease or condition, reduction of a symptom associated with the disease or condition, prevention or alleviation of a secondary disease or condition resulting from the occurrence of a primary disease or condition (e.g., atherosclerosis resulting from diabetes), and/or prevention of the disease or condition.

Examples of therapeutic proteins that can be produced using the method of the present invention include, but are not limited to, a protein having antimicrobial activity (discussed in detail above), a cytokine, or a protein or peptide drug. In one embodiment, the therapeutic protein is a protein which has some particular benefit in being expressed by a primary human macrophage. More particularly, such a therapeutic protein is preferably capable of modifying the macrophage, or an activity of the macrophage, in such a manner that a benefit is achieved. For example, as described above, the expression of an antimicrobial agent by a human primary macrophage is particularly beneficial because the macrophage is a primary cell type involved in the innate immune response against a broad spectrum of extracellular and intracellular pathogenic microorganisms and additionally, the macrophage is the natural host cell for Mycobacterium tuberculosis. Therefore, expression of an antimicrobial protein by a primary human macrophage can inhibit or prevent microbial cell growth or even kill the microbe, and thereby provide a significant therapeutic benefit to a human. To increase the expression of other proteins by the human primary macrophage may have similar benefits. Alternatively, the high efficiency with which the primary human macrophage can be transfected makes the cell an attractive host, cell for the in vitro production of virtually any protein that can be expressed by transfection of mRNA, and particularly, of many peptides. A preferred protein to produce using this method of the present invention is an antimicrobial protein as discussed above and including, but not limited to, defensins such as human β-defensin 2.

In a preferred embodiment, the therapeutic protein is expressed by the human primary macrophage in an amount effective to inhibit growth of a microorganism. In another preferred embodiment, the therapeutic protein is expressed by the human primary macrophage in an amount effective to substantially prevent growth of a microorganism. Examples of both of these embodiments are provided in the Examples section below. In another embodiment, the therapeutic protein is expressed by the human primary macrophage in an amount effective to kill at least a statistically significant portion of the microbes in a given population of microorganisms. Preferably, at least about 5%, and more preferably at least about 10%, and more preferably at least about 25%, and more preferably at least about 35%, and more preferably at least about 45%, and more preferably at least about 55%, and more preferably at least about 65%, and more preferably at least about 75%, and more preferably at least about 85%, and more preferably at least about 95% of the microbes in a given population of microorganisms are killed.

In an in vitro embodiment of this method, if desired, the therapeutic protein can be recovered from the primary human macrophage and/or the culture medium. The phrase “recovering the protein” refers to collecting the whole culture medium and/or cell containing the protein and need not imply additional steps of separation or purification. Proteins of the present invention can be purified using a variety of standard protein purification techniques, such as, but not limited to, affinity chromatography, ion exchange chromatography, filtration, electrophoresis, hydrophobic interaction chromatography, gel filtration chromatography, reverse phase chromatography, concanavalin A chromatography, chromatofocusing and differential solubilization.

Other embodiments of this method of the present invention, including detailed descriptions of mRNA (including amounts to be delivered, methods of delivery, preferred transfection efficiencies), antimicrobial proteins, human primary macrophages, liposome delivery vehicles, and methods of transfection, have been described in detail above with respect to the first described method of the present invention. The description above applies equally to this embodiment of the present invention and will not be reiterated here.

Another embodiment of the present invention relates to a method for treating and/or preventing a disease caused by a pathogenic microorganism in a human patient that is infected with, or susceptible to, respectively, infection with the microorganism. The method includes the step of transfecting human primary macrophages in the human patient with a composition comprising: (a) an isolated mRNA encoding a therapeutic protein; and, (b) a liposome delivery vehicle. The isolated mRNA is transfected at a concentration of at least about 0.5 μg mRNA per ml of liposome, is expressed by the human primary macrophage, and is effective to inhibit the growth of the microorganism in the patient. This method is useful for the treatment of any disease or condition which is associated with infection of a human host by any of the pathogenic microorganisms discussed above (i.e., those that can infect human hosts).

Therapeutic proteins useful in the methods of the present invention have been discussed previously above. Preferably, the mRNA encodes an antimicrobial protein, including a defensin protein and more particularly, human β-defensin 2. In one embodiment, the patient is infected with, or susceptible to infection with, Mycobacterium tuberculosis, which results in or can result in tuberculosis in the patient. In this embodiment, the therapeutic protein is preferably a defensin.

Preferably, the therapeutic protein is expressed by the human primary macrophage in an amount effective to inhibit growth of a microorganism. In another embodiment, the therapeutic protein is expressed by the human primary macrophage in an amount effective to substantially prevent growth of a microorganism.

Various embodiments of this method of the invention, including a description of the mRNA, liposome, host cells, liposome delivery vehicles, therapeutic proteins, methods of transfection and methods of delivery of the composition to a host have been described previously herein and apply equally to this embodiment of the invention.

As used herein, the phrase “protected from a disease” refers to reducing the symptoms of the disease; reducing the occurrence of the disease, and/or reducing the severity of the disease. Protecting a patient can refer to the ability of a composition of the present invention, when administered to a patient, to prevent a disease from occurring and/or to cure or to alleviate disease symptoms, signs or causes. As such, to protect a patient from a disease includes both preventing disease occurrence (prophylactic treatment) and treating a patient that has a disease (therapeutic treatment). Preferably, to treat a disease results in reduction of microbe growth in the patient to an extent that the patient no longer suffers discomfort and/or altered function resulting from or associated with infection by the microorganism. The term, “disease” refers to any deviation from the normal health of a mammal and includes a state when disease symptoms are present, as well as conditions in which a deviation (e.g., infection, gene mutation, genetic defect, etc.) has occurred, but symptoms are not yet manifested.

The following examples are provided for purposes of illustration and are not intended to limit the scope of the present invention.

### EXAMPLES

### Example 1

The following example describes the optimization of transfection efficiency for transfection of mRNA into macrophages using enhanced green fluorescent protein (eGFP).

Monocytes were isolated from human whole blood by centrifugation through Ficoll-Hypaque. The mononuclear cell layer was washed in RPMI 1640+saline, and the monocytes counted. Approximately 1×10⁶ monocytes were dispensed into the wells of 24 well plates (Falcon, Becton Dickinson), and allowed to adhere for one hour. The monolayers were then washed three times to remove non-adherent cells. The resulting cell monolayers consisted of <95% monocytes as determined by hydrolysis of the non-specific esterase substrate fluorescein di-acetate and epifluorescence microscopy. The few remaining non-monocytes appeared to be lymphocytes based on morphology. Monocyte monolayers were then cultured at 37° C. for eight days to allow for differentiation into macrophage-like cells prior to infection with M. tuberculosis (Erdman). Cells were placed at 100,000/well into 8 well chambered coverslips (Nalge-Nunc intl., Naperville, Ill.) and allowed to adhere for 2 hours in RPMI1640 including penicillin (0.05 units/ml), streptomycin (0.05 μg/ml), L-glutamine, and 10% autologous human serum. Non-adherent cells were then removed with 3 washes with warm PBS, and the medium replaced with antibiotic-free Macrophage-SFM (Gibco-BRL, Gaithersburg, Md.). The monocytes were then allowed to differentiate into macrophages for 6 to 7 days at 37° C., 5% CO₂.

Initially, mRNA encoding enhanced green fluorescent protein (eGFP) was used to determine the transfection efficiency and optimal mRNA/lipid ratio and concentration. To prepare the eGFP construct, a DNA fragment encoding enhanced GFP (eGFP) was amplified from the retroviral plasmid pMXI-eGFP (provided by Dr. Gary Nolan, Cleveland Clinic) using PCR primers which incorporated Xba-1 (5′) and Sac-1 (3′) restriction sites. The PCR product was digested with those two enzymes to mature the ends and cloned into the Sac-1 to Xba-1 sites of pSP64-poly A (Promega, Madison Wiss.). After amplification and purification from E. coli, the pSP64-eGFP-poly A plasmid was linearized at the end of the poly A addition tract using Eco-R1. Capped mRNA encoding e-GFP was made by in vitro transcription using the Message Machine kit (Ambion, Austin Tex.) according to a protocol supplied by the manufacturer. The DNA template was removed by treatment of the reactions with DNAase 1 for 30 minutes. The mRNA was purified by extraction with phenol:chloroform:isoamyl alcohol (Ambion, Austin Tex.), followed by removal of low molecular weight constituents by column chromatography over Sephadex G-50 spin columns (NICKspin columns, Pharmacia, Uppsala Sweden). The resulting mRNA had a A₂₆₀/A₂₈₀ ratio of approximately 1.95. The mRNA was stored at −70° C. until use.

Unless otherwise specified, for transfection of 1 well containing approximately 100,000 macrophage cells, 2 μg of eGFP mRNA was combined with 1 μg of Oligofectin G (Sequitur, Natik MA) in 0.2 mL of serum free, antibiotic free RPMI-1640 in a 5 ml polystyrene culture tube (Falcon, Becton Dickinson, San Jose, Calif.). The mixture was vortexed at high speed for 30 seconds, and then allowed to stand at 25° C. (room temperature) for 15 minutes. The macrophage monolayers were washed once with PBS, and the medium replaced with the mRNA/Oligofectin G complex, or yeast mRNA/Oligofectin G complex for controls in RPMI-1640. The cultures were then returned to the incubator for two hours, after which fetal bovine serum (FBS) was added to 10%. The cells were incubated for an additional 4 hours, and then fixed with neutral buffered formalin for 30 minutes at 4° C. After fixation, the cells were washed extensively with 1M glycine, pH 7.2 in order to inactivate residual formaldehyde and retard the development of autofluorescence. The fixed cells were then allowed to stand overnight at 4° C. in the dark to allow full oxidation of the e-GFP chromophore, which is essential for development of fluorescent properties.

The cells were examined and recorded using a Nikon Diaphot inverted microscope fitted with epifluorescence illumination and a CCD camera system (Nu200, Photometrics, Tucson, Ariz.). Fluorescence intensity was recorded during 0.3 second exposures with a gain setting of 4 using IP Lab spectrum software (Scanalytics, Vienna, Va.). Intensity was integrated within the region defined by the cell, and the average background of an area devoid of cells was subtracted.

Several different ratios of RNA to cationic lipid were initially tested (data not shown). The ratio which provided the best GFP expression at 2 μg/ml of RNA was tested at higher concentrations of RNA as well. Results achieved with 8 μg/ml of eGFP mRNA (300 μg/ml lipid), showed that greater than 90% of macrophages exhibited fluorescence, indicating successful penetration of the mRNA into the cytoplasm of most of the macrophages (data not shown). The average fluorescence intensity of the cells increased with the concentration of mRNA applied, up to 8 μg/ml. Increasing the mRNA concentration to 16 μg/ml did not result in a further increase. The frequency of eGFP expression exceeded levels reported for transfection of eGFP-encoding plasmids into macrophages by at least 40 fold, (>90% positive) than previously reported for plasmids (2% positive) (Lauth et al., Insect Biochem Mol Biol 28:1059-66, 1998; and Simoes et al., J Leukoc Biol 65:270-9, 1999).

To prepare mycobacterial suspensions, the mycobacterial lawn from the surface of Middlebrook 7H11 agar plates were collected when growth had reached mid-log phase. Mycobacteria were placed into 5 ml of macrophage SFM medium (Gibco) in 16×125 mm round-bottom borosilicate glass screw-cap culture tube with 8-10 3 mm glass beads (Fisher Scientific) and vortexed in pulses six times. Clumps of mycobacteria were allowed to settle at unit gravity for 45 minutes. Thus, supernatant containing a mainly single cell suspension was then transferred to a new tube and allowed to settle for an additional 30 minutes. The supernatant was then transferred to 16×125 mm flat-bottom borosilicate glass screw-cap culture tube (Fisher Scientific), and the number of bacterial cells was determined spectrophotometrically in a nephrometer (Becton Dickinson CrystalScan). Mycobacterial suspensions were diluted to an optical density of 1 McFarland unit/ml (1×10⁸ cells/ml).

The growth kinetics of M. tuberculosis can be reproducibly measured in monolayers of human MDM when infecting with low innoculum in tissue culture. These procedures were performed under biosafety level 3 (BSL-3) conditions in the Mycobacteriology Laboratory at National Jewish Medical and Research Center, Denver Colo. This laboratory has developed and standardized an in vitro system for testing anti-mycobacterial drugs (Mor et al., Antimicrob Agents Chemother 40:1482-5, 1996). This standardized procedure has been further developed to be used for a study of agents which may modulate macrophage activity (data not shown). Macrophage monolayers were infected by replacing medium with macrophage SFM containing the appropriate numbers of M. tuberculosis bacilli. Infection was allowed to continue for 1 hour, after which the monolayers were vigorously washed twice with RPMI 1640+saline, and incubated further in macrophage-SFM.

Results showed that infection with M. tuberculosis did not reduce transfection efficiency of eGFP mRNA into human MDM (data not shown).

### Example 2

The following example demonstrates the relative toxicities of mRNAs encoding GFP vs hBD-2.

Cationic lipids are known to be toxic to mammalian cells at high concentration (Freedland et al., Biochem Mol Med 59:144-53, 1996), as are defensins (Lichtenstein et al., Blood 68:1407-1410, 1986). The present inventors therefore sought to determine the maximum dose of GFP mRNA/oligofectin G complex which could be applied to the macrophages, and whether human β-defensin (hBD-2) mRNA had greater toxicity. Specifically, the inventors determined the ability of human monocyte-derived macrophages (MDM) to reduce MTT 24 hours after treatment with increasing concentrations of either GFP mRNA (produced as described in Example 1) or hBD-2 mRNA (see below) complexed with Oligofectin G.

hBD-2 cDNA was produced by RT-PCR using human tracheal epithelial cell mRNA as a template and published primer sequences (Harder et al., Nature 387:861, 1997, incorporated herein by reference in its entirety). The cDNA was cloned into the SMA I site of pBluescript. Templates for in vitro transcription of hBD-2 mRNA were made via PCR from the hBD-2 cDNA using an upstream oligo bearing a promoter for bacteriophage T7 RNA polymerase, and a downstream oligo bearing a 25 residue oligo dT extension for templated addition of a poly A tail to the in vitro transcript. In vitro transcription was carried out as described for the eGFP template (See Example 1). Macrophages were transfected with various amounts of hBD-2 mRNA combined with Oligofectin G as described above for eGFP (See Example 1).

Cell viability was measured by incubation of the cells with 5 mg/ml of 3-(4,5-dimethylthiazol-2-yl)-2,5-diphenyl tetrazolium bromide (MTT). After 24 hours the absorbance of reduced MTT was measured at 585 nm for macrophages treated with Oligofectin-G:hBD-2 mRNA: complex or Oligofectin G: eGFP mRNA complex. Cell viability was measured via reduction of MTT in at least 3 experiments. Results showed that reduction of MTT was not affected by the eGFP mRNA/cationic lipid complex until the concentration exceeded 32 μg/ml (data not shown). In contrast, complexes of hBD-2 mRNA and oligofectin G became toxic at 8 μg/ml (data not shown), indicating that the proteins produced by translation of the mRNAs differed in toxicity as predicted.

### Example 3

The following example demonstrates the production of hBD-2, and association with intracellular M. tuberculosis following mRNA transfection.

It was thought that the ability of hBD-2 to affect the growth of M. tuberculosis within macrophages would depend in part on the ability of the defensin protein to gain access to the mycobacteria. Therefore, immunocytochemistry was performed using a specific rabbit anti-human hBD-2 antiserum to determine if hBD-2 protein was produced following transfection of macrophages with hBD-2 mRNA, and to determine where in the macrophages the protein localized.

For immunocytochemistry, cells were grown on eight chamber slides, fixed in formalin at 4° C., and washed in 1M glycine. Immunohistochemistry was carried out as described (Yount et al., J Biol Chem 274:26249-58, 1999), using specific hBD-2 antibody (a gift of T. Ganz) or non-immune serum, and visualized using the Vector ABC kit (Vectorlabs). Acid-fast staining of mycobacteria was carried out using Difco TB auramine-rhodamine stain according to the protocol supplied by the manufacturer (Becton Dickinson Microbiology Systems, Sparks, Md.). hBD-2-transfected macrophages were infected with M. tuberculosis as described in Example 1.

Using auramine-O staining and epifluorescence microscopy, the presence of M. tuberculosis was visualized. The results showed a lack of specific staining of M. tuberculosis infected, hBD-2 mRNA treated macrophages with preimmune rabbit serum (control; data not shown). Infected macrophages which were transfected with mRNA encoding eGFP and stained with anti-hBD-2 antiserum showed a similar lack of staining for hBD-2 (data not shown). However, when the specific anti-hBD-2 antiserum was used with infected macrophages which had been treated with hBD-2 mRNA 24 hours earlier, specific staining is observed within the macrophages (data not shown). The staining pattern is punctuate and reminiscent of bacilli. Counterstain of the sample with auramine-O revealed that the cytoplasmic structures stained with anti-hBD-2 antiserum were acid fast bacilli. These data indicate that hBD-2 was produced by the macrophages following transfection with hBD-2 mRNA, and that the hBD-2 was able to gain access to the intracellular mycobacteria. However, not all of the acid-fast bacilli were positive for hBD-2 after treatment with mRNA.

### Example 4

The following example demonstrates a dose response of inhibition ofM. tuberculosis growth in hBD-2 transfected macrophages.

Since the hBD-2 produced by the macrophages was determined to bind to the intracellular mycobacteria, it was next determined if sufficient hBD-2 could be produced by the macrophages following mRNA transfection to inhibit the growth of M. tuberculosis. For this experiment, macrophage monolayers were infected with M. tuberculosis (Erdman) as described in Example 1 at a 10:1 ratio of bacilli to macrophages. In the present inventors' hands, this ratio results in infection of approximately 30% of the macrophages. One hour after infection, the monolayers were treated with increasing concentrations of hBD-2 mRNA, or eGFP mRNA complexed with Oligofectin G ranging from 0.5 μg/ml to 8 μg/ml. The monolayers were then incubated at 37° C., 5% CO₂ for four days, after which the monolayers were lysed 1 ml of 0.25% SDS for 10 minutes. The lysates were diluted with 7H9 medium to neutralize the SDS, and spread onto Middlebrook 7H11 plates for colony growth for 21 days at 37° C. to determine the number of mycobacterial CFU remaining.

The results demonstrated that growth of M. tuberculosis was inhibited in monolayers treated with 0.5 μg/ml of hBD-2 mRNA, but not with e-GFP mRNA. Growth of M. tuberculosis in the monolayers was prevented by treatment with 2 μg/ml or more of hBD-2 mRNA, but was enhanced by treatment with the same concentrations of eGFP mRNA. Therefore, hBD-2 mRNA treatment resulted in concentration dependent inhibition of mycobacterial growth, with minimal inhibitory concentration (MIC) of approximately 2 μg/ml, or 20 nM.

### Example 5

The following example demonstrates extended duration of M. tuberculosis growth inhibition following single administration of hBD-2 mRNA.

Following determination that macrophages transfected with hBD-2 mRNA could inhibit the growth of M. tuberculosis, the present inventors tested the duration of the growth inhibition. hBD-2 mRNA was administered as above at concentrations of 2, 4, and 8 μg/ml complexed with oligofectin G as described in Example 1. Monolayers infected with M. tuberculosis as described in Example 1 were lysed and mycobacterial CFU determined by growth on 7H11 plates 0, 2, 5, and 7 days after infection. The results showed that treatment with hBD-2 mRNA resulted in reduction of CFU between days 0 and 2, whereas mycobacterial numbers remained constant in monolayers treated with eGFP mRNA, and increased in untreated monolayers. Mycobacteria increased by 2-3 fold between days 2 and 7 in cells treated with hBD-2 mRNA, but increased approximately 10 fold in cells treated with e-GFP mRNA. Mycobacteria in monolayers which were untreated increased 50 fold overall between days 0 and 7, while numbers of mycobacteria in the hBD-2 mRNA treated cultures did not exceed those present at the beginning of the experiment. Therefore, the mycobacterial growth inhibition mediated by macrophages treated with a single addition of hBD-2 mRNA lasted for at least 7 days. Upon microscopic inspection of the monolayers, cells treated with hBD-2 mRNA appeared much healthier, with few signs of infection at the end of 7 days, whereas untreated cells or those which received mRNA encoding eGFP showed extensive cytopathology, with many dead cells by day 7 (data not shown).

Several cationic lipid formulations were examined in these studies, many of which showed efficacy in delivering exogenous mRNA to the cytoplasm. However, Oligofectin G was effective at a lower concentration, and was less toxic to the macrophages relative to Lipofectamine, DOTAP, Lipofectin, or DMRIE/Cholesterol (data not shown). The toxicity of the hBD-2 mRNA/cationic lipid complexes was greater than for the GFP mRNA/cationic lipid complex. This may be due to the reported toxicity of hBD-2 for mammalian cells (Lichtenstein et al., Blood 68:1407-1410, 1986 Lichtenstein et al., Blood 68:1407-1410, 1986) rather than the lipid portion of the complex. Toxicity of the GFP/cationic lipid complex observed above 32 μg/ml of RNA is most likely due to the complex rather than the mRNA or the lipid, as the components of the complex were either not toxic in the concentration range tested (the GFP mRNA), or were only toxic at much greater concentrations (Oligofectin G). Such toxicity has been reported for other nucleic acid/cationic lipid complexes (Freedland et al., Biochem Mol Med 59:144-53, 1996).

Growth of intracellular mycobacteria was inhibited as a result of transfecting mRNA encoding hBD-2, but not GFP, in a dose dependent manner. The IC₅₀ for hBD-2 mRNA was 2 μg/ml (20 nM), which was approximately 4 fold less than the dose which was toxic to the macrophages. It is unclear whether the IC₅₀ represents transfection of 50% of the macrophages with sufficient mRNA to completely inhibit growth of the bacilli, or whether all of the macrophages were transfected with a similar amount of hBD-2 mRNA, which was sufficient to mediate 50% inhibition of growth. The inhibition of growth was robust, and remained evident for at least 7 days of culture with a single addition of hBD-2 mRNA.

The number of viable mycobacteria in the macrophages declined by approximately 50% in the first 24 hours after infection when the macrophages were treated with hBD-2 mRNA (See Examples 4 and 5). These data imply that a true bactericidal effect could potentially be achieved by administering the mRNA to the cultures at 2 day intervals. This is consistent with other data we have gathered using luciferase mRNA as the reporter for murine macrophages (not shown). The dosing schedule may lend itself to further optimization for maximum anti-mycobacterial activity of hBD-2 mRNA, as may the structure and chemistry of the mRNA itself. The native mRNA encoding hBD-2 contains relatively long 5′ and 3′ untranslated regions (UTR) predicted to have extensive secondary structure of unknown function, but which maintain extensive homology with other β-defensins (Diamond and Bevins, Clinic. Immunol. and Immunopathol. 88:221-225, 1998). Stability and translational efficiency may be improved by replacement of the native UTRs with those from β-globin, which is a very stable and efficiently translated mRNA in most cell types (Kisich et al., J Immunol 163:2008-16, 1999). The mRNA may also be further stabilized by alteration of the 2′OH groups (Heidenreich et al., J Biol Chem 269:2131-8, 1994), and replacing some of the bridging phosphate groups with phosporothioate groups (Heidenreich et al., Antisense Nucleic Acid Drug Dev 6:111-8, 1996) without abolishing translational activity (Aurup et al., Nucleic Acids Res 22:4963-8, 1994).

In summary, the results of Examples 1-5 demonstrate that cultured primary human macrophages can be efficiently transfected with mRNA encoding potentially therapeutic proteins. The efficiency of transfection observed following delivery of an eGFP mRNA/Oligofectin G complex (>90%), was approximately 40 fold greater than had previously been reported for cultured human macrophages using electroporation or lipoplex mediated delivery of DNA reporter vectors (Simoes et al., J Leukoc Biol 65:270-9, 1999; Van Tendeloo et al., Gene Ther 5:700-7,1998; Weir and Meltzer, Cell Immunol 148:157-65, 1993).

While various embodiments of the present invention have been described in detail, it is apparent that modifications and adaptations of those embodiments will occur to those skilled in the art. It is to be expressly understood, however, that such modifications and adaptations are within the scope of the present invention, as set forth in the following claims.

What is claimed is:

1. A method to inhibit the growth of a microorganism, comprising transfecting a human cell with an isolated mRNA encoding a protein having antimicrobial biological activity, wherein said human cell expresses said protein and thereby inhibits the growth of a microorganism when said microorganism contacts said human cell;
wherein said human cell is a natural host cell for said microorganism or naturally contacts said microorganism when a human is infected with said microorganism.

2. The method of claim 1, wherein said human cell does not naturally express said protein.

3. The method of claim 1, wherein said human cell is a primary macrophage.

4. The method of claim 3, wherein said primary human macrophage resides in lung tissue.

5. The method of Claim l, wherein said microorganism is a pathogenic microorganism.

6. The method of claim 1, wherein said microorganism is selected from the group consisting of a bacterium, a fungus, a virus, a protozoa and a parasite.

7. The method of claim 6, wherein said bacterium is a bacterium selected from the group consisting of: a spirochete, a mycobacterium, a Gram (+) cocci, a Gram (−) cocci, a Gram (+) bacillus, a Gram (−) bacillus, an anaerobic bacterium, a rickettsias, a Chlamydias and a mycoplasma.

8. The method of claim 6, wherein said bacterium is a mycobacterium.

9. The method of claim 6, wherein said microorganism is a fungus selected from the group consisting of: a pathogenic yeast, a mold and a dimorphic fungus.

10. The method of claim 6, wherein said microorganism is an enveloped virus.

11. The method of claim 1, wherein said protein is a defensin.

12. The method of claim 1, wherein said protein is a β-defensin.

13. The method of claim 1, wherein said protein is a human β-defensin 2.

14. The method of claim 1, wherein said step of transfecting includes transfecting a liposome containing said mRNA into said human cell.

15. The method of claim 1, wherein said human cell is transfected with a concentration of at least about 0.5 μg/ml of said mRNA.

16. The method of claim 1, wherein said human cell is transfected with a concentration of at least about 2 μg/ml of said mRNA.

17. The method of claim 1, wherein at least about 1 pg of said protein having antimicrobial biological activity is expressed per mg of total cellular protein per μg of nucleic acid transfected into said cell.

18. The method of claim 1, wherein the transfection efficiency of said method is at least about 50%.

19. The method of claim 1, wherein the transfection efficiency of said method is at least about 75%.

20. The method of claim 1, wherein the transfection efficiency of said method is at least about 90%.

21. The method of claim 1, wherein said human cell is transfected with an amount of defensin protein that is not toxic to said cell.

22. The method of claim 1, wherein said human cell expresses said defensin intracellularly.

23. The method of claim 1, wherein said step of transfecting is performed ex vivo.

24. A method of expression of a therapeutic protein in a human primary macrophage, comprising transfecting said human primary macrophage with a composition comprising:
a. an isolated mRNA encoding a therapeutic protein; and,
b. a liposome delivery vehicle;
wherein said isolated mRNA is transfected at a concentration of at least about 0.5 μg/ml mRNA;
wherein said therapeutic protein is expressed by said human primary macrophage.

25. The method of claim 24, wherein said mRNA is transfected at a concentration of at least about 1 μg/ml mRNA.

26. The method of claim 24, wherein said mRNA is transfected at a concentration of at least about 2 μg/ml mRNA.

27. The method of claim 24, wherein the transfection efficiency of said method is at least about 50%.

28. The method of claim 24, wherein the transfection efficiency of said method is at least about 75%.

29. The method of claim 24, wherein the transfection efficiency of said method is at least about 90%.

30. The method of claim 24, wherein at least about 1 pg of said therapeutic protein is expressed per mg of total cellular protein per μg of nucleic acid transfected into said cell.

31. The method of claim 24, wherein said liposome delivery vehicle comprises cationic lipids.

32. The method of claim 24, wherein said mRNA encodes a protein that is not naturally expressed by said primary human macrophage.

33. The method of claim 24, wherein said mRNA encodes an antimicrobial protein.

34. The method of claim 24, wherein said mRNA encodes a defensin protein.

35. The method of claim 24, wherein said mRNA encodes human β-defensin 2.

36. The method of claim 24, wherein said therapeutic protein is expressed by said human primary macrophage in an amount effective to inhibit growth of a microorganism.

37. The method of claim 24, wherein said therapeutic protein is expressed by said human primary macrophage in an amount effective to substantially prevent growth of a microorganism.

38. The method of claim 24, wherein said step of transfecting is performed ex vivo.

39. A method for treating a disease caused by a pathogenic microorganism in a human patient that is infected by said pathogenic microorganism, comprising transfecting human primary macrophages in said human patient with a composition comprising:
(i) an isolated mRNA encoding a therapeutic protein; and,
(in) a liposome delivery vehicle;
wherein said isolated mRNA is transfected at a concentration of at least about 0.5 μg/ml mRNA;
wherein said therapeutic protein is expressed by said human primary macrophage, and wherein said protein is expressed so that growth of said microorganism is inhibited.

40. The method of claim 39, wherein said pathogenic microorganism is Mycobacterium tuberculosis, wherein said therapeutic protein is a defensin, and wherein said disease is tuberculosis.

41. The method of claim 39, wherein said mRNA encodes an antimicrobial protein.

42. The method of claim 39, wherein said mRNA encodes a defensin protein.

43. The method of claim 39, wherein said mRNA encodes human β-defensin 2.

44. The method of claim 39, wherein said therapeutic protein is expressed by said human primary macrophage in an amount effective to inhibit growth of a microorganism.

45. The method of claim 39, wherein said therapeutic protein is expressed by said human primary macrophage in an amount effective to substantially prevent growth of a microorganism.
